(19) United States
(12) Patent Application Publication
(10) Pub. No.: US 2025/0177575 A1
(43) Pub. Date: Jun. 5, 2025

(54) OPTICAL SENSOR WITH SINGLE-WALLED CARBON NANOTUBE IN A POLYMER MATRIX

(71) Applicant: Research Foundation of the City University of New York, New York, NY (US)

(72) Inventors: Ryan Williams, New York, NY (US); Steven Nicoll, Cedarhurst, NY (US); Zachary Cohen, New York, NY (US); David Alpert, Brooklyn, NY (US); Adam Weisel, New York, NY (US)

(21) Appl. No.: 18/964,811

(22) Filed: Dec. 2, 2024

Related U.S. Application Data
(60) Provisional application No. 63/605,001, filed on Dec. 1, 2023.

(51) Int. Cl. A61K 49/00; C01B 32/159; C08L 1/26; C12N 15/11

(52) U.S. Cl. CPC A61K 49/0019; A61K 49/0054; C01B 32/159; C08L 1/26; C12N 15/11; C08L 2203/02

(57) ABSTRACT

An optical sensor with a DNA-wrapped single-walled carbon nanotube (SWCNT) disposed within a polymer matrix of methylcellulose or sulfonated methylcellulose. A hydrogel may be formed in vivo that is optically sensitive to an analyte.

### CROSS-REFERENCE TO RELATED APPLICATIONS

This application claims priority to, and is a non-provisional of, U.S. Patent Application 63/605,001 (filed Dec. 1, 2023), the entirety of which is incorporated herein by reference.

### STATEMENT OF FEDERALLY SPONSORED RESEARCH OR DEVELOPMENT

This invention was made with Government support under grant numbers R35GM142833 and U54CA132378 awarded by the National Institute of Health. The government has certain rights in the invention.

### REFERENCE TO A SEQUENCE LISTING

This application contains a Sequence Listing in computer readable form. The computer readable form is incorporated herein by reference. The computer readable file is named Seqeuence.xml and was created on Nov. 28, 2024 (2 kB size).

### BACKGROUND OF THE INVENTION

Single-walled carbon nanotubes (SWCNTs), cylindrical allotropes of carbon, have garnered considerable research interest in recent years for their exceptional mechanical, electrical, and optical properties. In particular, semiconducting SWCNTs exhibit near-infrared fluorescence, making them well-suited to use as transducers in optical biosensors. SWCNTs exhibit large stokes shifts, do not photobleach, and fluoresce in the tissue-transparent window. This has enabled implanted SWCNT-based biosensors to retain their fluorescence properties for at least 300 days in vivo, and at depths of 5.5 cm ex vivo. SWCNT-based biosensors have been developed for a variety of analytes, including metals, nitrous oxide, glucose, chemotherapeutics, auxins, insulin and other hormones, neurotransmitters, oligonucleotides, riboflavin, fibrinogen, growth factors, amyloid beta, cytokines, lipid accumulation diseases, cardiac biomarkers, cancer biomarkers, and more.

Hydrogels are a diverse and customizable class of materials. Their chemical properties may be tuned to control their mechanical properties, porosity, biocompatibility, and other properties relevant to injection at numerous physiological locations. They have such diverse application as platforms for drug and cell delivery, cell recruitment, and as scaffolds for tissue engineering. Their high degree of tunability is important for SWCNT implants in particular, as such a device must facilitate analyte diffusion into the gel while preventing SWCNT diffusion out of the gel.

To date, several studies have demonstrated SWCNT-based biosensor implants in living animals. SWCNTs can be administered intravenously, which results in SWCNT uptake in the liver Kupffer cells, allowing sensing within those cells. For biosensing in other locations, the majority of studies surgically implanted SWCNTs in either sealed dialysis membranes or hydrogels under anesthesia. Surgical implantation makes biosensing possible at any location but is, by nature, an invasive procedure. Even when the nanotubes are dispersed in pre-gelled polymers they must be administered through trochar devices.

An improved SWCNT system for detecting select analytes is therefore desired. The discussion above is merely provided for general background information and is not intended to be used as an aid in determining the scope of the claimed subject matter.

### SUMMARY

This disclosure provides an optical sensor with a DNA-wrapped single-walled carbon nanotube (SWCNT) disposed within a polymer matrix of methylcellulose or sulfonated methylcellulose. A hydrogel may be formed in vivo that is optically sensitive to an analyte.

In a first embodiment, a composition of matter is provided comprising a single-walled carbon nanotube (SWCNT) wrapped with (1) a single-stranded DNA with 100 or fewer bases or (2) a single-stranded RNA with 100 or few bases; and a polymer matrix comprised of 1-5% (m/m) of a cellulose material in aqueous buffer, wherein the cellulose material is methylcellulose or sulfonated methylcellulose.

In a second embodiment, a hydrogel is provided that is produced by mixing (1) a redox initiator (2) a single-walled carbon nanotube (SWCNT) that is wrapped with (1) a single-stranded DNA or (2) single-stranded RNA and (3) a polymer matrix comprised of 1-5% (m/m) of a cellulose material in aqueous buffer, wherein the cellulose material is methylcellulose or sulfonated methylcellulose.

In a third embodiment, a method is provided comprising steps of: injecting into an animal a two-component composition, wherein a first component comprises a redox initiator and a second component comprises a single-walled carbon nanotube (SWCNT) wrapped with (1) a single-stranded DNA or (2) a single-stranded RNA and a polymer matrix comprised of 1-5% (m/m) of a cellulose material in aqueous buffer, wherein the cellulose material is methylcellulose or sulfonated methylcellulose, the injecting occurring such that the first component mixes with the second component within the animal; and irradiating the hydrogel with electromagnetic radiation; recording a change in intensity or a shift in wavelength of an emitted wavelength that is produced form the step of irradiating, wherein the change in intensity or the shift in wavelength is indicative of the presence of an analyte within the animal.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a representative fluorescence spectra of the nanotube sensor in all hydrogels tested.

FIG. 2A illustrates sensor response (center wavelength) to MgCl₂ and NaHCO₃ in alginate-acrylamide hydrogels. MgCl₂ and NaHCO₃ induced significant wavelength shifting versus control (p=4×10⁻⁶ and 0.02 respectively). N=6.

FIG. 2B illustrates sensor response (intensity) to MgCl₂ and NaHCO₃ in alginate-acrylamide hydrogels. NaHCO₃, but not MgCl₂, caused a significant intensity change (p=0.04). N=6.

FIG. 3A depicts sensor response (center wavelength) to MgCl₂ and NaHCO₃ in a chitosan-based hydrogel. NaHCO₃ did not cause shifting at any timepoint; MgCl₂ induced significant shifting on hours 1, 2, 3, and 24 (p=8×10⁻⁴, 0.03, 0.01, and 0.02 respectively).

FIG. 3B depicts sensor response (intensity) to MgCl₂ and NaHCO₃ in a chitosan-based hydrogel. No significant differences were observed in intensities. N=3.

FIG. 4A shows sensor response (center wavelength) to MgCl₂ in MC-based hydrogels. P-values for 3% MC shifts on days one through three were 0.001, 4×10⁻¹⁰, and 6×10⁻⁵ respectively; p-values for 2% MC on days two and three were 2×10⁻⁶ and 2×10⁻¹² respectively.

FIG. 4B shows sensor response (intensity) to MgCl₂ in MC-based hydrogels. P-values for 3% MC intensity changes on days one through three were 0.004, 9×10⁻⁷, and 0.048 respectively; p-values for 2% MC on days two and three were 3×10⁻⁴ and 0.006 respectively. N=4-5.

FIG. 5A illustrates sensor response (center wavelength) to NaHCO₃ in MC-based hydrogels. The 2% sensor gel exhibited a slight but significant shift at hours one and 48 (p=0.004 and 0.001 respectively). N=3-4.

FIG. 5B illustrates sensor response (intensity) to NaHCO₃ in MC-based hydrogels. The 3% sensor gel system only showed a significant intensity-based response on the second day (p=0.047). N=3-4.

FIG. 6A shows MgCl₂ Quantification (shift) in MC Based Hydrogels. Fits use the Hill model; r²≥0.99 for all fits. Shift-derived K_{D} values are 44.1 and 60.3 μM for 3% and 2% gels respectively.

FIG. 6B shows MgCl₂ Quantification (intensity) in MC Based Hydrogels. Fits use the Hill model; r² ≥0.99 for all fits. Intensity change-derived K_{D} values are 16.9 and 17.1 μM for 3% and 2% gels respectively.

FIG. 7A depicts sensor response (center wavelength) to MgCl₂ and BSA in sulfonated MC Hydrogels. Significant wavelength (p<0.0001) differences were observed for all groups on days 1 and 6.

FIG. 7B depicts sensor response (intensity) to MgCl₂ and BSA in sulfonated MC Hydrogels. Significant intensity differences (p<0.0001) were observed for all groups on days 1 and 2 (except for MgCl₂ on day 2 in 2% MC-SO₃). N≥4.

FIG. 8A shows quantification (shifts (7.5) chirality) of BSA in 3% MC-SO₃ hydrogels and solution.

FIG. 8B shows quantification (shifts (9.4) chirality) of BSA in 3% MC-SO₃ hydrogels and solution.

FIG. 8C shows quantification (intensity changes of 7.5 chirality) of BSA in 3% MC-SO₃ hydrogels and solution.

FIG. 8D shows quantification (intensity changes of 9.4 chirality) of BSA in 3% MC-SO₃ hydrogels and solution.

FIG. 9A illustrates quantification (shifts of (7.5) chirality) of doxorubicin in 3% MC-SO₃ hydrogels and solution.

FIG. 9B illustrates quantification (shifts of (9.4) chirality) of doxorubicin in 3% MC-SO₃ hydrogels and solution.

FIG. 9C illustrates quantification (intensity of (7.5) chirality) of doxorubicin in 3% MC-SO₃ hydrogels and solution.

FIG. 9D illustrates quantification (intensity of (9.4) chirality) of doxorubicin in 3% MC-SO₃ hydrogels and solution.

FIG. 10A shows fluorescence spectra (655 nm excitation) from (GT)₁₅-SWCNT encapsulated in 3% MC-SO₃ hydrogels subcutaneously injected across 61 days.

FIG. 10B shows fluorescence spectra (730 nm excitation) from (GT)₁₅-SWCNT encapsulated in 3% MC-SO₃ hydrogels subcutaneously injected across 61 days.

FIG. 11A shows DOX wavelength response of sensor implants in vivo. Representative autofluorescence-subtracted spectra of implants in vivo with 655 nm excitation and

FIG. 11B shows fluorescence peaks (data as dots, models as lines) from the same mouse just before and two days after DOX administration of the (7.6) chirality.

FIG. 11C shows shifting over time of implants in control and experimental groups for (7.6) chirality.

FIG. 11D shows DOX wavelength response of sensor implants in vivo. Representative autofluorescence-subtracted spectra of implants in vivo with 730 nm excitation.

FIG. 11E shows fluorescence peaks (data as dots, models as lines) from the same mouse just before and two days after DOX administration of the (9.4) chirality.

FIG. 11F shows shifting over time of implants in control and experimental groups for (9.4) chirality.

FIG. 12 is a graph showing DOX ratiometric response of sensor implants in vivo. Percent change in ratio of (9.4) fluorescence intensity to (7.6) fluorescence intensity. In the experimental group, the (9.4) chirality got dimmer relative to the (7.6) chirality. This relative dimming was not observed for the control group. p=0.007 and 0.02 at hours 24 and 48 respectively.

FIG. 13A shows a rheological comparison (elasticity modulus) of 3% MC-SO₃ gels with and without sensors. No significant differences were observed.

FIG. 13B shows a rheological comparison (gelation time) of 3% MC-SO₃ gels with and without sensors. Gelation time is defined as point where the derivative of the elasticity modulus <1 Pa. No significant differences were observed.

FIG. 14 depicts BSA content of 2% and 3% MC gels post-topping. MC gels topped with 1×PBS or BSA (600 μM) in 1×PBS were degraded by cellulase. A BCA assay was then used to quantify the BSA content of the degraded gels, revealing that gels topped with 1×PBS had no BSA content whereas 2% and 3% gels topped with BSA contained 140±30 and 160±20 μM BSA respectively. p<0.0001

### DETAILED DESCRIPTION OF THE INVENTION

This disclosure provides a methacrylated methylcellulose—SO₃ (MC-SO₃) composition that is useful as a functional platform for single-walled carbon nanotubes (SWCNT)-based biosensor encapsulation. This disclosure demonstrates the potential of injectable hydrogels as a non-invasive means of implanting SWCNT biosensors. This disclosure also demonstrates that a salt (MgCl₂) and a representative small-molecule drug, doxorubicin (DOX), diffuse through the polymer matrix. Bovine serum albumin (BSA), a 66.5 kDa protein, was able to permeate the gel and interact with SWCNTs. This confirms the pore size of the disclosed MC-SO₃ is not an obstacle to large biomarker diffusion. These experiments have also demonstrated that relatively precise analyte quantification is possible with this method of SWCNT encapsulation and delivery. Logistic models of (GT)₁₅-SWCNT fluorescence response as a function of analyte concentration showed R² values as high as 0.99. (GT)₁₅ is GTGTGTGTGTGTGTGTGTGTGTGTGTGTGT (SEQ ID NO: 1).

For detection of the model globular protein analyte BSA, both response sensitivity and magnitude were increased in MC-SO₃ compared to solution. The system also exhibits promising stability and functionality in vivo; implanted in mice, the sensors were shown to fluoresce for at least 61 days, and to discriminate mice injected with DOX from those injected with phosphate buffered silane (PBS) as a control.

(GT)₁₅-SWCNTs were prepared as previously described (Sci Adv 2018, 4 (4), eaaq1090 and ECS Journal of Solid State Science and Technology 2022, 11 (10)). Briefly, in a 1.5 mL microcentrifuge tube, approximately 0.5 mg HiPCO SWCNTs (NanoIntegris, Boisbriand, Quebec, Canada) and (GT)₁₅ single-stranded DNA (10 g/L, Integrated DNA Technologies, Coralvile, IA, USA) suspended in 1×PBS (phosphate-buffered saline, Sigma-Aldrich, St. Louis, MO, USA) were added to a final oligonucleotide:SWCNT mass ratio of 2:1. 1×PBS was added to bring the final volume to 500 μL. To achieve aqueous dispersion of SWCNTs by (GT)₁₅, the solution was sonicated (1 hr., 40% amplitude) in an ice bath by a VCX 750 (Sonics & Materials, Inc., Newtown, CT, USA) fitted with a 2 mm stepped-microtip. The suspension was then ultracentrifuged (58,000×g, 1 hr., 4° C.) in an Optima MAX-XP (Beckman Coulter, Indianapolis, IN, USA) to separate residual catalyst, amorphous carbon, and any partially suspended SWCNTs from the (GT)₁₅-SWCNTs. The top 75% of the solution was stored at 4° C. until further use.

Prior to an experiment, excess (GT)₁₅ was removed by filtration. An aliquot (100-375 μL) of the (GT)₁₅-SWCNT solution was loaded into a 100 kDa molecular weight cutoff filter (Millipore Sigma, St. Louis, MO, USA) and centrifuged (14,000 RPM, 15 min, 4° C.) in a Sorvall ST8R centrifuge (Thermo Fisher Scientific, Waltham, MA, USA). The filtrate was discarded, the contents of the filter were resuspended in 1×PBS (500 μL), and centrifugally filtered again. (GT)₁₅-SWCNTs remaining in the filter were resuspended in 1×PBS (100-200 μL).

In other embodiments, single-stranded DNA other than (GT)₁₅ is used, provided the DNA has 100 or fewer nucleotides or 50 or fewer nucleotides. In some embodiments, a single-stranded RNA is used provided the RNA has 100 or fewer nucleotides or 50 or fewer nucleotides. The DNA or RNA may be conjugated to an antibody. The polymer matrix generally comprises 1-5% (wt) of methylcellulose or sulfonated methylcellulose in an aqueous buffer. Any suitable aqueous buffer may be used (e.g. PBS, tris-buffered saline (TBS), 3-(N-morpholino) propanesulfonic acid (MOPs), 4-(2-hydroxyethyl)-1-piperazineethanesulfonic acid (HEPES), citrate buffers, tris-EDTA (TE) etc.). The DNA-SWCNT is present in the polymer matrix at a concentration from, for example, 0.5 mM to 500 nm, 1 mM to 100 mM or from 1 mM to 10 mM.

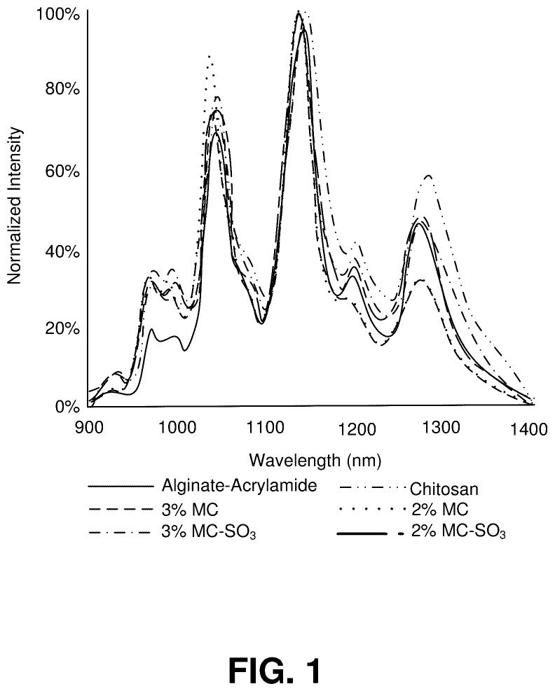

Referring to FIG. 1, the fluorescence of SWCNT-based nanosensors were evaluated in six hydrogels.

TABLE 1

| | |
|---|---|
| 1 | Alginate-acrylamide |
| | (Mater Sci Eng C Mater Biol Appl 2019, 95, 409-421) |
| 2 | 3% (wt) MC |
| 3 | 3% (wt) MC-SO₃ |
| 4 | Chitosan |
| 5 | 2% (wt) MC |
| 6 | 2% (wt) MC-SO₃ |

SWCNT fluorescence was detected in all gels tested. As expected, nanotube fluorescence was found in relatively hydrophilic gels (e.g., chitosan) and was red-shifted relative to those in relatively hydrophobic gels (e.g., methylcellulose).

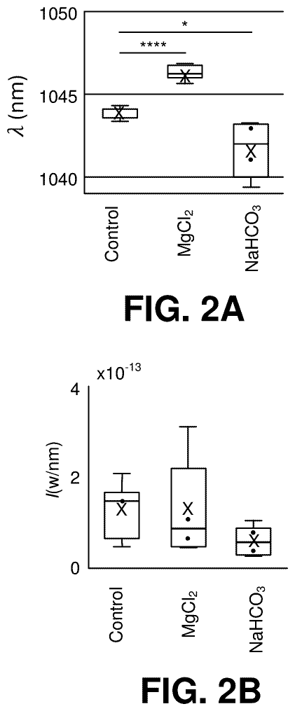

Referring to FIG. 2A and FIG. 2B, the first hydrogel tested was a previously described alginate-acrylamide copolymer solution that crosslinks at body temperature. The copolymer is comprised primarily of poly(N-isopropylacrylamide) grafted to an alginate backbone for extra rigidity. It was selected for these experiments because alginate gels are commonly used in experiments with SWCNTs, while crosslinking at body temperature is appealing for injection. As a naturally-occurring polysaccharide, alginate is highly abundant and biocompatible. It is also known for forming gels relatively easily, especially via the addition of a divalent crosslinker.

The sensing ability of (GT)₁₅-SWCNTs in the alginate-acrylamide gel was evaluated using magnesium chloride (MgCl₂, 5 M) and sodium bicarbonate (NaHCO₃, 1 M) as representative analytes. MgCl₂ and NaHCO₃ were chosen for this initial response evaluation because their small hydrodynamic radii and high charge were hypothesized to better facilitate diffusion and detection. MgCl₂ was of particular interest as previous studies have demonstrated good detection of divalent cations by SWCNTs. After SWCNT-containing alginate-acrylamide gels were incubated with the analytes for one hour at body temperature, NaHCO₃ was found to cause partial quenching (intensity decreased by 53±54% versus control) and blue-shifting (−2.3±1.8 nm) (FIG. 2B). MgCl₂ only caused shifting (2.3±0.6 nm, FIG. 2A), though this shift was more consistent than that induced by NaHCO₃.

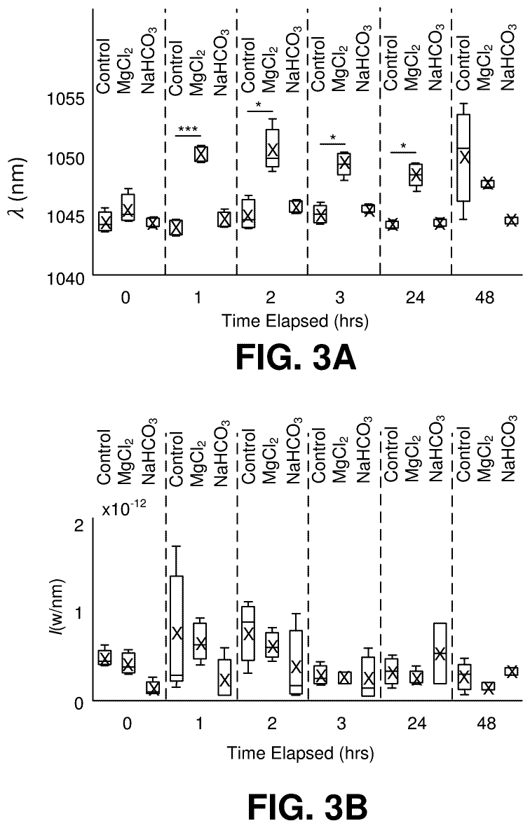

Referring to FIG. 3A and FIG. 3B, sensor encapsulation was investigated in a chitosan gel, also known to crosslink at body temperature, whose mechanical properties were previously shown to improve upon incorporation of graphene oxide (Front Chem 2018, 6, 565). Similar to alginate, chitosan was an appealing polymer because of its abundance and biocompatibility. The sensing ability of (GT)₁₅-SWCNTs in the chitosan gel was again evaluated using MgCl₂ (5 M) and NaHCO₃ (1 M) as model analytes. While neither caused an intensity-based response (FIG. 3B), MgCl₂ induced red-shifting (6.1±1.1 nm) which plateaued after an hour (FIG. 3A) and then decreased by a third (to 4.2±1.4) nm after the first day. Minimal response was found to NaHCO₃, likely because the gel is itself basic.

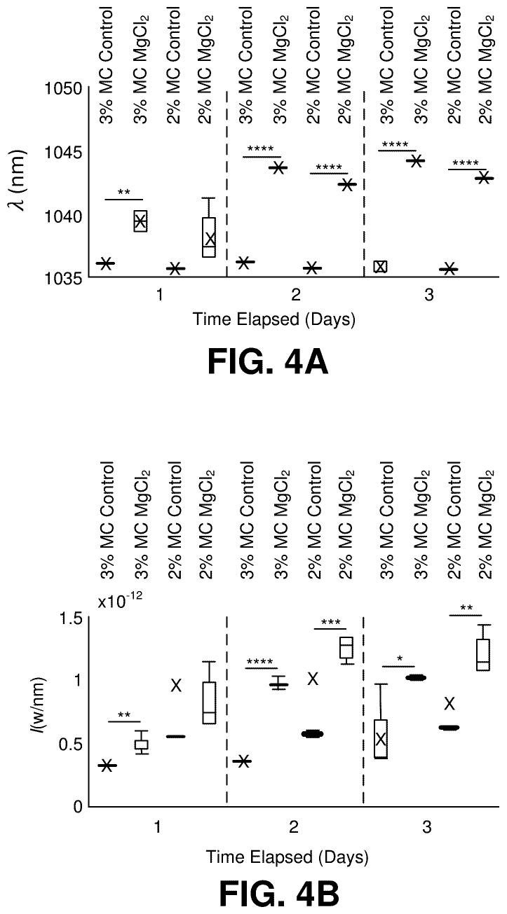

Referring to FIG. 4A and FIG. 4B, while both the chitosan and alginate-acrylamide gels gave acceptable results for model analyte detection, they are known to be less mechanically robust than desirable for long-term implantation. Accordingly, a gel based on methacrylated methylcellulose (MC) with demonstrated biocompatibility and mechanical properties facilitating long-term in vivo stability was investigated. This particular gel was originally investigated for tissue engineering applications, and therefore is already well-suited to clinical translation. Additionally, cellulose is the most abundant polysaccharide and is therefore easy to source (Hydrogels Based on Natural Polymers, 2020; pp 17-47).

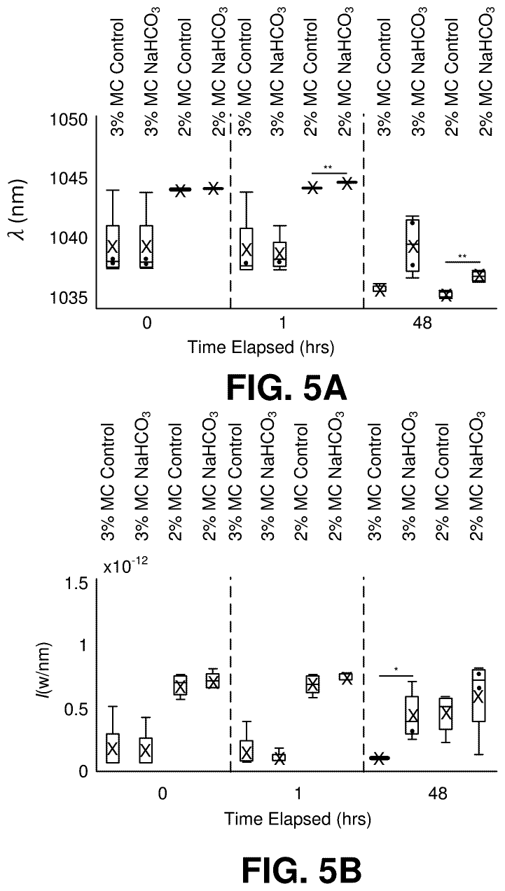

MC gels were initially tested at polymer concentrations of 2% and 3%. While 3% MC gels were previously shown to have optimal mechanical properties for implantation as an adipose tissue mimic, 2% MC gels were speculated to better facilitate analyte diffusion because of their higher porosity. Though neither MC-SWCNT formulation responded to MgCl₂ (5 M) by the first day, large red shifts were observed two days into the experiment (FIG. 4A and FIG. 4B, 7.5±0.2 nm and 6.6±0.2 nm for 3% and 2% gels respectively). While 2% MC was not found to perform better than 3% MC for MgCl₂ detection, a similar experiment found that NaHCO₃ (1 M) only elicited a response from the 2% system, inducing red shifting two days after exposure (FIG. 5A, 1.6±0.5 nm). The 3% sensor gel system only showed a significant intensity-based response on the second day (p=0.047) (FIG. 5B).

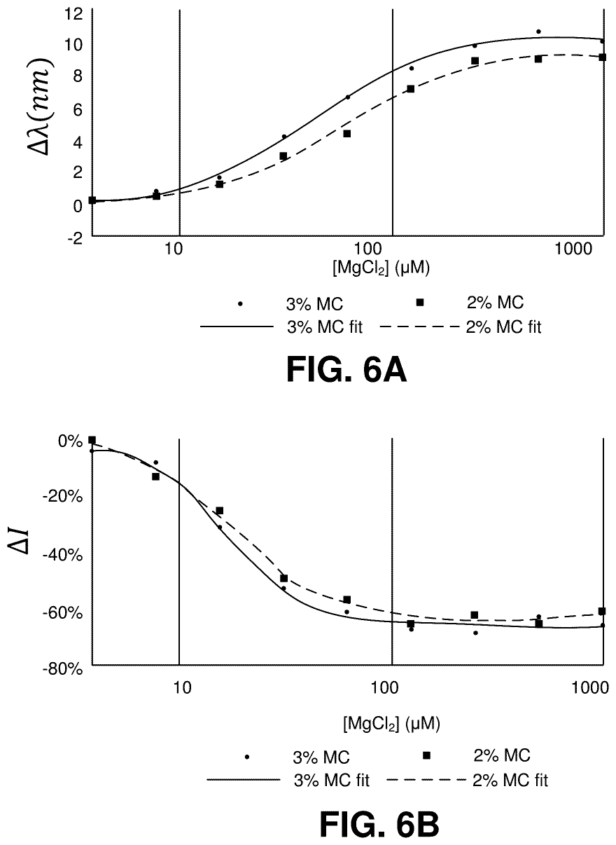

Referring to FIG. 6A and FIG. 6B, because the MC-SWCNT system demonstrated the most substantial responses and has the most desirable mechanical properties, this system was further explored to evaluate the breadth of MC's potential as an in vivo sensor delivery platform. To evaluate the system's capacity for analyte quantification across a range of concentrations, MC-SWCNT samples were topped with MgCl₂ from 4 to 1000 μM. Concentration-dependent shift—(FIG. 6A) and intensity-based (FIG. 6B) responses were observed after two days and fit to a Hill model. Interestingly, the intensity-based response was found to have higher sensitivity, with a fit-derived K_{D} of about 20 μM, compared to about 50 μM for the shift response. The decoupled nature of shifting and intensity responses may allow for analyte quantification across a broader range of concentrations than would otherwise be possible.

To potentially increase the response rate while maintaining the MC-SWCNT system's mechanical properties, sulfonated versions were also investigated. As MC gels are largely hydrophobic, it was hypothesized that increasing polymer hydrophilicity by charge functionalization would better facilitate the diffusion of salts and proteins. Sulfonate groups were added to the MC polymers by replacing some of the methacrylate groups prior to gelation. This was accomplished using a previously described gel preparation procedure (Lin, H. A. Development and Functional Evaluation of Injectable Cellulose-based Hydrogels as Nucleus Pulposus Replacements for Intervertebral Disc Repair. Ph.D., The City College of New York, United States—New York, 2018). SWCNTs encapsulated in sulfonated 3% MC exhibited fluorescence across the entire 149-day duration of a stability assessment experiment (see FIG. S2 in U.S. provisional application 63/605,001). Following gelation of the 3% sulfonated methylcellulose formulation with encapsulated SWCNTs, its visco-elastic properties were maintained in comparison to controls with only minor reductions in both elasticity modulus and gelation time (see FIG. 13A and FIG. 13B).

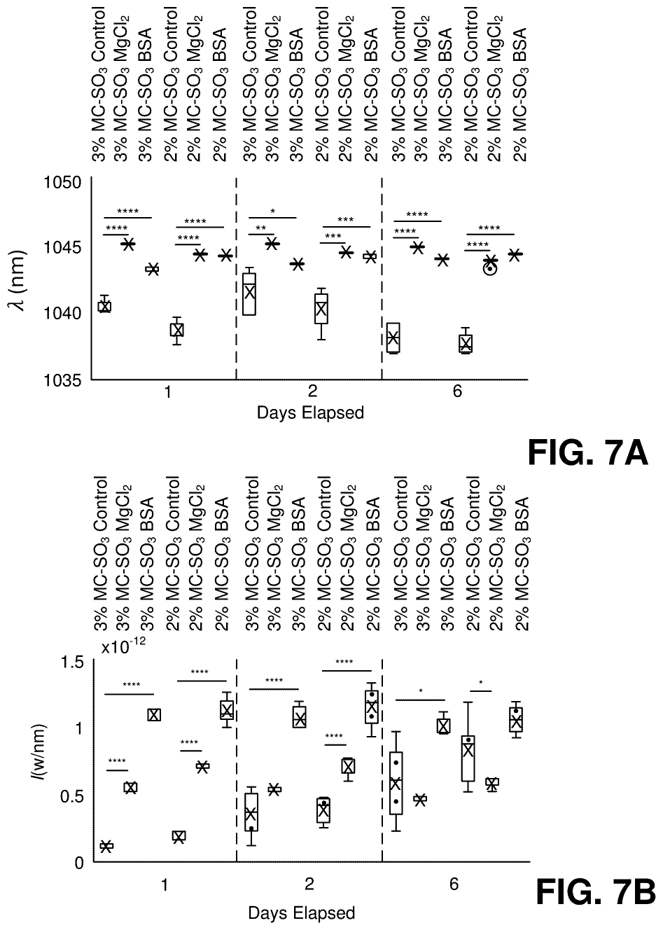

2% and 3% concentrations of the sulfonated methylcellulose (MC-SO₃) gel with (GT)₁₅-SWCNTs were evaluated for their response to MgCl₂ (5 M) and bovine serum albumin (BSA, 600 μM). BSA was introduced as a representative large molecular weight globular protein, though it should be noted that non-invasive sensing of albumin has particularly important clinical implications in liver, renal, and cardiovascular disorders. Indeed, a SWNCT-based nanosensor paint has previously been reported for the pre-clinical detection of albuminuria (Nat Commun 2019, 10 (1), 3605). Both the sulfonated systems were found to have significant shift- and intensity-based responses to MgCl₂ or BSA one day after incubation, an improved detection speed compared to the non-sulfonated devices (FIG. 7A and FIG. 7B). At this timepoint, the 3% gel-encapsulated SWCNTs responded to BSA with a shift and intensity change of +2.7±0.6 and +660±42% respectively; for the 2% system wavelength and intensity responses of +5.5±0.7 and +360±50% were observed. While wavelength shifts were maintained throughout the experiment, intensity differences moderated between days 2 and 6, largely due to increases in control intensity rather than diminution of analyte response.

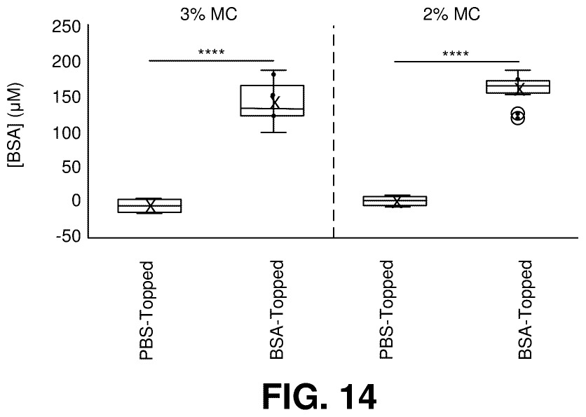

BSA diffusion into MC gels was confirmed by a BCA assay (see U.S. FIG. 14). Analyte-containing topping solutions were removed and replaced with a solution of cellulase. Protein quantification was then performed on the degraded gels to determine the amount of BSA present, finding that gels exposed to BSA contained 130-250 μM of the protein, and that there were no clear differences in diffusion between the 2% and 3% gels.

Additionally, the ability of the 3% MC-SO₃-SWCNT system to maintain its BSA response over a two-week period was evaluated. The 2% MC formulation was not used because sulfonation had a greater impact on response speed and the 3% concentration better approximates tissue. Significant wavelength- and intensity-based responses were observed across the entire experiment duration (see U.S. provisional application 63/605,001). The wavelength-based response diminished across the experiment duration (from +8.9±0.5 nm to +2.9±1 nm), whereas the intensity response plateaued after three days (at +62%±10% of the control's intensity). While the wavelength-based response can largely be attributed to red-shifting of the experimental group, the intensity-based response derives from intensity changes in the control group (see U.S. provisional application 63/605,001).

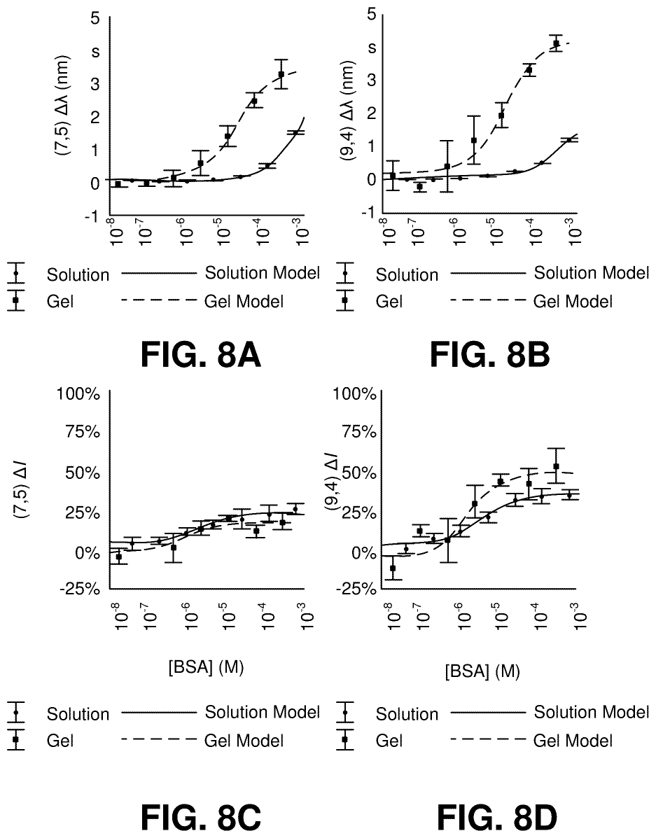

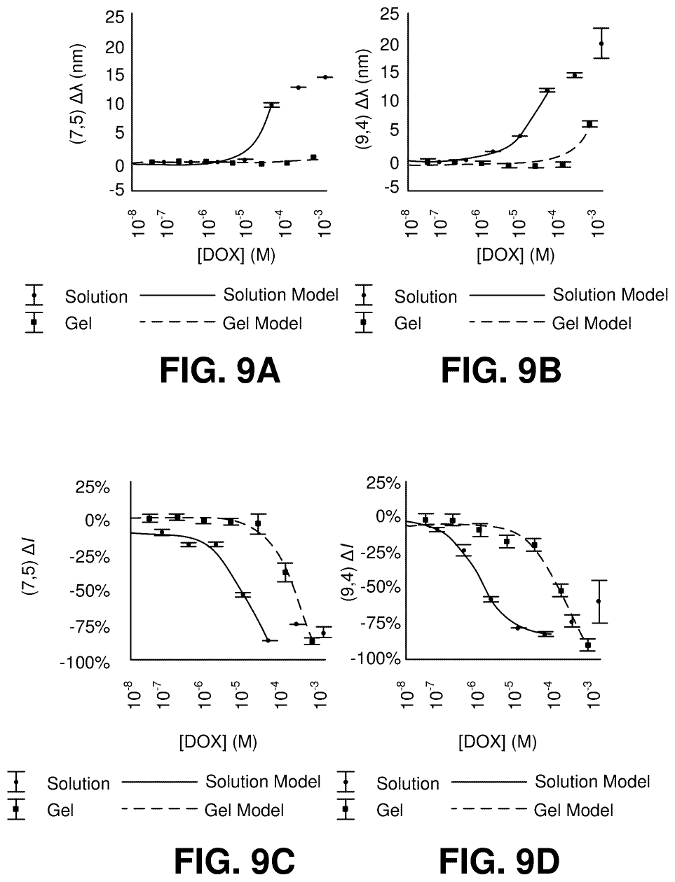

Referring to FIG. 8A, FIG. 8B, FIG. 8C and FIG. 8D, concentration-response curves were obtained in gel and solution to evaluate the effect of 3% MC-S03 encapsulation on (GT)15-SWCNT response dynamics at 37° C. Responses were measured at analyte concentrations from 10 nM to 10 mM. These experiments were executed using a high-throughput 96-well plate format, enabled by a custom-built near-IR plate-reader spectrophotometer. Responses were fit to a logistic trend (see U.S. provisional application 63/605,001). Responses to albumin were tested while separately introducing a new analyte, doxorubicin (DOX). DOX is a small molecule anthracycline chemotherapeutic used as a front-line therapy in several cancers. Previous studies demonstrated SWCNT-based fluorescent sensors implanted within a dialysis membrane for DOX pharmacokinetic monitoring in mice (Nat Biomed Eng 2017, 1 (4), 0041).

Whereas 3% MC-SO₃ encapsulation increased BSA sensitivity (FIGS. 8A-8D) and response magnitude versus solution conditions, it also decreased DOX sensitivity (FIGS. 9A-9D). For BSA, the shift-derived K_{D} decreased by one to two orders of magnitude for every chirality except (8,7) (e.g., 920 to 16.7 μM for the (7.5) chirality). Interestingly, intensity change-derived K_{D} values did not change appreciably. In contrast, gel encapsulation increased the intensity change-derived DOX K_{D} by one to two orders of magnitude (e.g., 10.7 to 134. μM for the (7.5) chirality). However, the MC-SO₃ system better fit the expected trend across the tested concentration range, likely due to DOX aggregation at 10 μM in 1×PBS.

For both analytes, response curve goodness-of-fit was decreased after gel encapsulation, likely because of variance in gel volumes expelled by the dual-barrel syringe. In the case of BSA, this increased variance is partially offset by an increased shift magnitude, leading to a higher signal-to-noise ratio. Response times also increased; whereas the maximum responses were achieved by the first hour in solution, the maximum BSA and DOX responses were observed after 24 and 48 hours respectively.

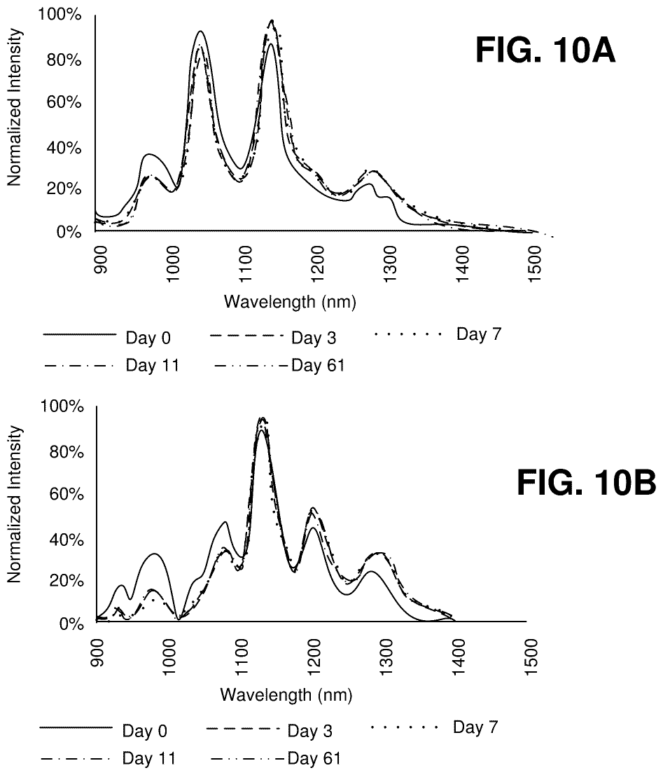

Referring to FIG. 10A and FIG. 10B, following in vitro validation of this system, the small molecule chemotherapeutic doxorubicin was detected in live animals. First, a mouse was injected with MC-SO₃-encapsulated SWCNTs to evaluate the stability of the device in vivo. Clear near-infrared fluorescence emanating from gel-encapsulated SWCNTs was observed on each day investigated, up to two months post-injection. Minimal variations in emission spectra were noted upon excitation at 655 and 730 nm (FIG. 10A and FIG. 10B, respectively).

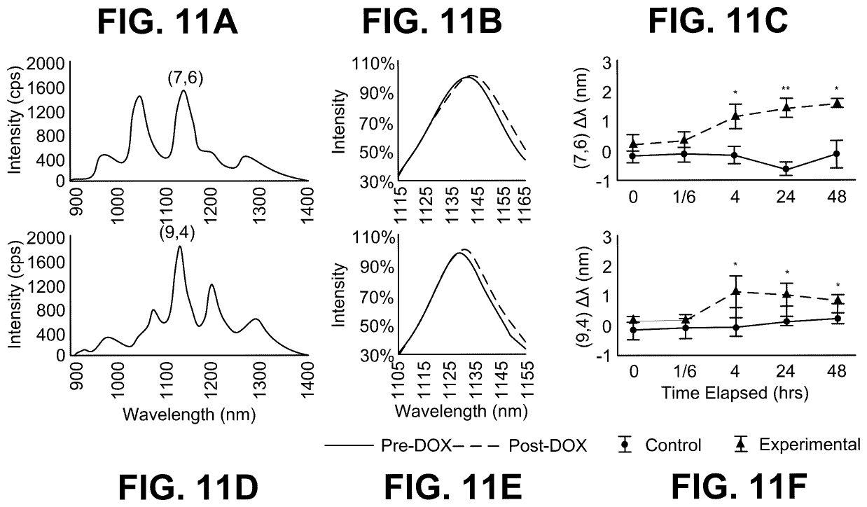

Referring to FIG. 11A-11F, to evaluate the function of the MC-SO₃-SWCNT formulation in vivo, it was injected into six mice. After the implants were allowed to crosslink (approximately 15 minutes), the mice were anesthetized with isoflurane, and baseline spectra were acquired using an IRina NIR-II spectral probe (Photon, Etc.) with 655 and 730 nm excitation lasers coupled to an InGaAs detector. Mice were then subcutaneously dosed with DOX or 1×PBS as control at four sites around the implant location (n=3). Fluorescence spectra were again acquired immediately, 10 minutes, 4 hours, 24 hours, and 48 hours post-dosing.

FIG. 11A is a NIR fluorescent spectra of nanotubes inside the MC hydrogel in a mouse after excitation with a 655 nm laser. FIG. 11B is a close-up of the spectra from 1115-1165 nm, showing a change in control and doxorubicin addition. FIG. 11D shows the NIR fluorescent spectra of nanotubes inside the MC hydrogel in a mouse after excitation with a 730 nm laser. FIG. 11E shows the same change as FIG. 11B.

Analyses of these spectra showed that the implants exhibited a shifting-based response to DOX in vivo (FIG. 11C and FIG. 11F). DOX caused the (7.6) and (9.4) chiralities to shift (experimental minus control) by 1.3±0.5 and 1.2±0.6 nm respectively by the four-hour timepoint, whereas the relatively dim (8.6) chirality only showed a significant response after 48 hours. Shifts at the 48-hour timepoint for the (7.6), (9.4), and (8.6) chiralities were, 1.7±0.5, 0.6±0.2 and 1.1±0.3 nm respectively. The (7.5) chirality was also bright enough to be accurately fit but exhibited no response.

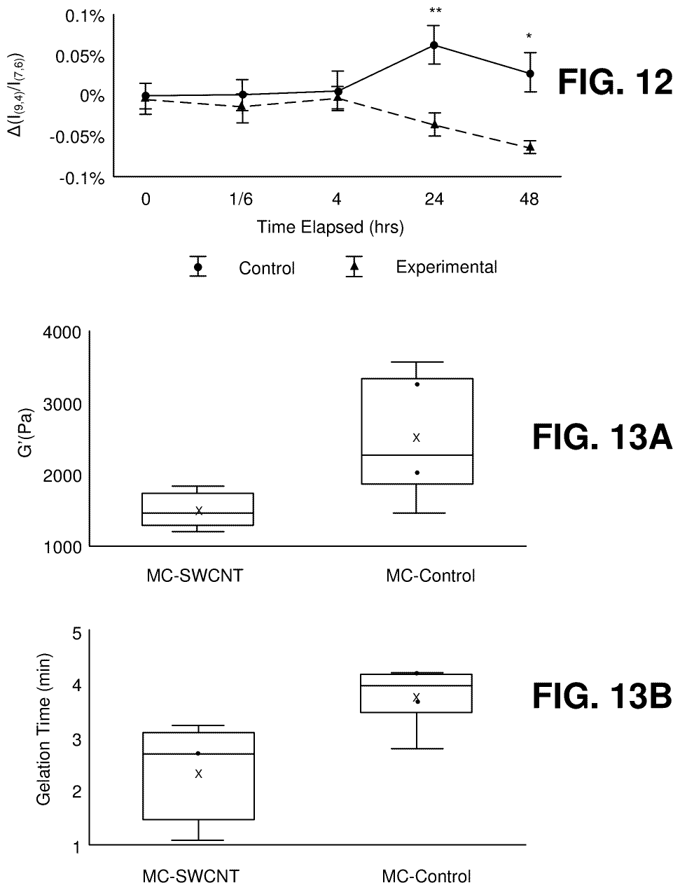

While it was not possible to observe quenching because of high variation in both experimental and control group intensities (attributable to differences in sensor positioning across measurements), DOX did cause a ratiometric intensity response (FIG. 12). In the experimental group, the (9.4) chirality got dimmer relative to the (7.6); the same effect was not observed in the control group.

### Methods

### Sensor Preparation

(GT)₁₅-SWCNTs were prepared as previously described (Sci Adv 2018, 4 (4), eaaq1090 and ECS Journal of Solid State Science and Technology 2022, 11 (10)). Briefly, in a 1.5 mL microcentrifuge tube, approximately 0.5 mg HiPCO SWCNTs (NanoIntegris, Boisbriand, Quebec, Canada) and (GT)₁₅ single-stranded DNA (10 g/L, Integrated DNA Technologies, Coralvile, IA, USA) suspended in 1×PBS (phosphate-buffered saline, Sigma-Aldrich, St. Louis, MO, USA) were added to a final oligonucleotide:SWCNT mass ratio of 2:1. 1×PBS was added to bring the final volume to 500 μL. To achieve aqueous dispersion of SWCNTs by (GT)₁₅, the solution was sonicated (1 hr., 40% amplitude) in an ice bath by a VCX 750 (Sonics & Materials, Inc., Newtown, CT, USA) fitted with a 2 mm stepped-microtip. The suspension was then ultracentrifuged (58,000×g, 1 hr., 4° C.) in an Optima MAX-XP (Beckman Coulter, Indianapolis, IN, USA) to separate residual catalyst, amorphous carbon, and any partially suspended SWCNTs from the (GT)₁₅-SWCNTs. The top 75% of the solution was stored at 4° C. until further use.

Prior to an experiment, excess (GT)₁₅ was removed by filtration. An aliquot (100-375 μL) of the (GT)₁₅-SWCNT solution was loaded into a 100 kDa molecular weight cutoff filter (Millipore Sigma, St. Louis, MO, USA) and centrifuged (14,000 RPM, 15 min, 4° C.) in a Sorvall ST8R centrifuge (Thermo Fisher Scientific, Waltham, MA, USA). The filtrate was discarded, the contents of the filter were resuspended in 1×PBS (500 μL), and centrifugally filtered again. (GT)₁₅-SWCNTs remaining in the filter were resuspended in 1×PBS (100-200 μL).

The resultant solution was characterized by absorbance spectroscopy as previously described (Sci Adv 2018, 4 (4), eaaq1090, ECS Journal of Solid State Science and Technology 2022, 11 (10)). Briefly, a visible absorbance spectrum was acquired from the (GT)₁₅-SWCNTs using a V-730 UV-VIS spectrophotometer (JASCO, Easton, MD, USA). Concentration was determined using an empirically derived extinction coefficient of 0.036 L mg⁻¹ cm⁻¹ for the absorption minimum at ˜630 nm.

### Fluorescence Spectrum Acquisition & Analysis

Final (GT)₁₅-SWCNT concentrations were 1 mg/L across all experiments. For experiments using the NS MiniTracer (Applied NanoFluorescence, TX, USA), samples were excited using a 50 mW 638 nm laser with 1-3 s exposure time. For experiments using the ClaIR IR plate reader (Photon Etc., Montreal, Quebec, Canada), samples were excited at 655 and 730 nm (sequentially) for 0.5 s at 1.75 W. For in-vivo experiments using the IRina in vivo NIR II spectral probe (Photon Etc.), spectra were acquired with de-focused 1 W laser excitation at 655 and 730 nm (sequentially), 1 s exposures, 1×2 binning, manual dark subtraction, and manual autofluorescence subtraction. Autofluorescence was modeled as exponential decay.

Fluorescence peaks were assigned to chiralities based on optical characterization in the literature. To determine the center wavelength and intensity of the (7.5) E₁₁ peak, the 24 points closest to the emission maximum around 1035-1045 nm were fit to a pseudo-Voigt model using custom MATLAB code. Fits of other chiralities were performed similarly, using consistent numbers of points for each peak (14 points for less prominent peaks, 28 points for broader peaks). Fits of binned spectra were performed using half as many data points. All analyses presented used fits deemed to be of sufficiently high quality (R²≥0.9).

Magnesium Chloride (MgCl₂) and Sodium Bicarbonate (NaHCO₃) Detection in Alginate-Acrylamide Gels

(GT)₁₅-SWCNT-containing (1.0 mg/L) alginate-acrylamide gels were prepared by modifying a previously published procedure (Mater Sci Eng C Mater Biol Appl 2019, 95, 409-421). Sodium alginate (116.7 mg, Thermo Fisher Scientific) and n-isopropylacrylamide (1524.94 mg, Thermo Fisher Scientific) were added to a glass vial containing (GT)₁₅-SWCNTs in 1×PBS (1 mg/L, 20 mL). The vial's contents were dissolved by slow magnetic stirring until visibly homogenous. Ascorbic acid (40 mg, Sigma-Aldrich) was added to the vial, which was then vortexed. Ammonium persulfate (50 mg, Thermo Fisher Scientific) was added to the vial, which was vortexed again and allowed to sit at room temperature for 15 minutes before being transferred to storage (4° C.).

1 mL of the resultant solution was transferred to 18 cuvettes, which were covered with parafilm and incubated in a water bath (0.5 hr., 40° C.) to gel the alginate-acrylamide copolymer. Fluorescence spectra were acquired from each sample using an NS MiniTracer (1 s exposure). Samples were then divided into three groups (n=6) and topped with 1 mL of either 1×PBS, magnesium chloride (MgCl₂, 5 M, Thermo Fisher Scientific), or sodium bicarbonate (NaHCO₃, 1 M, Thermo Fisher Scientific). Fluorescence spectra were again acquired using an NS MiniTracer (1 s exposure) after one hour of covered incubation in the water bath.

MgCl₂ and NaHCO₃ Detection in Chitosan Gels

(GT)₁₅-SWCNT-containing (1.5 mg/L) chitosan gels were prepared by modifying a previously published procedure (Front Chem 2018, 6, 565). Deionized water (5955.5 μL) and acetic acid (4.5 μL, Sigma-Aldrich) were added to a glass vial containing chitosan (200 mg, Sigma-Aldrich). The resulting mixture was dissolved by magnetic stirring at 40° C. for at least an hour. To this mixture glycerol 2-phosphate (3 M, Thermo Fisher Scientific) and (GT)₁₅-SWCNTs (3.75 mg/L) in deionized water (4 mL) were added. The resultant solution was slowly mixed magnetically for at least an hour.

1 mL of the resultant solution was transferred to each of nine cuvettes. The samples were covered with parafilm and gelled by incubation in a water bath (40° C., 1 hr). Fluorescence spectra were acquired using an NS MiniTracer (3 s exposure). The samples were then split into three groups and topped with 1 mL of deionized water, MgCl₂ (5 M), or NaHCO₃ (1 M). Fluorescence spectra were again acquired after one hour of covered incubation in the water bath. This procedure was repeated for a total of six samples per group.

### Methylcellulose-SWCNT Gel Preparation

(GT)₁₅-SWCNT-containing MC gels were prepared by modifying a previously published procedure (Carbohydr Polym 2015, 134, 497-507). Briefly, methylcellulose (Sigma-Aldrich) was methacrylated via the esterification of monomeric hydroxyl groups with methacrylic anhydride (Sigma-Aldrich) and then lyophilized for storage. Methacrylated MC was then dissolved in 1× Dulbecco's phosphate buffered saline (Thermo Fisher Scientific) containing (GT)₁₅-SWCNTs (1 mg/L). For sulfonated gels, 2-sulfoethyl methacrylate (PolySciences, Warminster, PA) was added to a final concentration of 5 mM. The resultant polymer-nanotube solution was then split in half to add redox initiators; to one half was added ammonium persulfate (final concentration 10 mM) and to the other was added an equimolar amount of ascorbic acid (Sigma-Aldrich). These solutions were then transferred to a dual barrel syringe fitted with a mixing tip (such that each barrel contained polymers, nanotubes, and one of the redox initiators). Gels were cast by extrusion through the mixing tip. Examples of suitable redox initiators include ascorbic acid, tert-butyl hydroperoxide (TBHP), ammonium iron (III) sulfate, sodium sulfite, hydrogen peroxide, etc. The redox initiators also function as a crosslinking agent.

### MgCl₂ Detection in Methylcellulose Gels

2% and 3% MC gels containing (GT)₁₅-SWCNTs (1.0 mg/L) were cast into pre-weighed cuvettes (250-500 mg gel per cuvette). After 30 minutes, cuvettes were weighed again and gels were topped with 1×PBS as control or MgCl₂ (5 M) in 1×PBS and then covered with parafilm. Solution volumes were 1 μL per mg gel. Fluorescence spectra were acquired using an NS MiniTracer (3 s exposure) immediately, every hour for the next three hours, and every day for the next three days. Samples were stored at 37° C. between measurements.

### NaHCO₃ Detection in Methylcellulose Gels

2% and 3% MC gels containing (GT)₁₅-SWCNTs (1.0 mg/L) were cast into cuvettes (250-500 mg gel per cuvette). After 30 minutes of incubation, gels were topped with 1×PBS (1 mL) as control or with NaHCO₃ (1 M, 1 mL) and then covered with parafilm (n=4). Fluorescence spectra were acquired with an NS MiniTracer (1 s exposure) immediately upon topping, after one hour, and after two days. Samples were stored at 37° C. between measurements.

### MgCl₂ Quantification in MC Gels

2% and 3% MC gels containing (GT)₁₅-SWCNTs (1.0 mg/L) were cast into pre-weighed cuvettes (250-500 mg gel per cuvette, 20 cuvettes total). After 30 minutes, cuvettes were weighed. For each MC concentration group (n=5-6), one cuvette was topped with 1×PBS as a control, and the nine remaining cuvettes were topped with MgCl₂ solutions in 1×PBS whose concentrations ranged from 1 M to 3.9 mM, decreasing by half. Solution volumes were 1 μL per mg gel. Cuvettes were covered with parafilm and stored at 37° C. between measurements. Fluorescence spectra were acquired with an NS MiniTracer (3 s exposure) 1, 2, and 6 days after gels were topped.

### MgCl₂ and Bovine Serum Albumin Detection in Sulfonated Methylcellulose Gels

2% and 3% MC and MC-SO₃ gels containing (GT)15-SWCNTs (1 mg/L) were cast into 54 pre-weighed cuvettes (250-500 mg gel per cuvette). After 30 minutes, cuvettes were weighed, and topped with 1×PBS as control, MgCl₂ (5 M), or bovine serum albumin (BSA, 600 μM) and then covered with parafilm (n=5-6). Solution volumes were 1 μL per mg gel. Fluorescence spectra were acquired using an NS MiniTracer (10 s exposure) 1-, 2-, and 6-days post-topping. Samples were stored at 37° C. between measurements. Separately, a set of identical experiments were performed to monitor BSA detection up to two weeks (n=7) with spectra acquired at 1, 2, 3, 4, 7, 8, and 14 days.

### BSA Quantification in Sulfonated Methylcellulose Gels

3% MC-SO₃ gels containing (GT)₁₅-SWCNTs (1 mg/L) were cast into a 96-well plate (150±50 μL per well, 24 wells). After 30 minutes, fluorescence spectra were acquired using a ClaIR IR plate-reader, then these wells were topped with solutions of BSA in 1×PBS (600, 120, 24, 4.8, 0.96, 0.192, 0.0384, and 0 μM; 150 μL; 3 wells per concentration). Fluorescence spectra were acquired immediately after topping, 1, 2, 3, 4, 5, 24, and 48 hours thereafter.

Solutions of (GT)₁₅-SWCNTs in 1×PBS (2 mg/L) were added to other wells in the same plate (150 μL per well, 21 wells). Fluorescence spectra were acquired before the addition of BSA solutions in 1×PBS (240, 48, 9.6, 1.92, 0.384, 0.0768, and 0 μM; 150 μL; 3 wells per concentration). Another set of wells was filled with (GT)₁₅-SWCNTs (1 mg/L) and BSA (600 μM) in 1×PBS (300 μL, 3 wells). Fluorescence spectra were acquired immediately, 1, 2, 3, 4, 5, 24, and 48 hours thereafter.

### Doxorubicin Quantification in Sulfonated Methylcellulose Gels

3% MC-SO₃ gels containing (GT)₁₅-SWCNTs (1 mg/L) were cast into a 96-well plate (150±50 μL per well, 24 wells). After 30 minutes, fluorescence spectra were acquired using a ClaIR IR plate-reader, then these wells were topped with solutions of doxorubicin (DOX) in 1×PBS (1000, 200, 40, 8, 1.6, 0.32, 0.064, and 0 μM; 150 μL; 3 wells per concentration). Fluorescence spectra were acquired immediately after topping, 1, 2, 24, and 48 hours thereafter.

Solutions of (GT)₁₅-SWCNTs in 1×PBS (2 mg/L) were added to other wells in the same plate (150 μL per well, 21 wells). Fluorescence spectra were acquired before the addition of DOX solutions in 1×PBS (200, 40, 8, 1.6, 0.32, 0.064, and 0 μM; 150 μL; 3 wells per concentration). Another set of wells was filled with (GT)₁₅-SWCNTs (1 mg/L) and DOX (1000 μM) in 1×PBS (300 μL, 3 wells). Fluorescence spectra were acquired immediately, 1, 2, 24, and 48 hours thereafter.

### Animal Studies

Studies in animals were performed in 4-6 week old female SKH1-Elite mice (Crl:SKH1-Hr^{hr}; Charles River, Wilmington, MA). Animals were housed under standard light/dark (12/12) conditions with ad libitum access to food and water. All experiments were approved by the Institutional Animal Care and Use Committee of The City College of New York.

### SWCNT-Gel Fluorescence Stability In Vivo

To evaluate the suitability of 3% MC-SO₃ as a SWCNT injection platform, a mouse was injected subcutaneously in the dorsal region with sterile 3% MC-SO₃ containing (GT)₁₅-SWCNTs. After 30 minutes, the mouse was anesthetized with isoflurane and spectra were acquired using an IRna NIR II spectral probe. Spectra were again acquired in this fashion after 3, 7, 11, and 61 days.

DOX Detection in vivo. To evaluate the response of (GT)₁₅-SWCNTs in 3% MC-SO₃ to DOX in vivo, ten mice were dorsally injected with the sensor-containing gel using a dual barrel syringe for subcutaneous injection. Fluorescence spectra were acquired using an IRna in vivo NIR II spectral probe from each mouse under anesthesia 30 minutes post-injection, after which they were immediately subcutaneously dosed with 10 mg/kg DOX in 1 mL 1×PBS or 1 mL 1×PBS as control (n=3). As mice masses ranged from 18-24 g, doses ranged from 330 to 440 nmol (330-440 μM). Doses were administered in quarters at four locations spaced around the implant site (4×250 μL). Spectra were acquired immediately, 10 minutes, 4 hours, 24 hours, and 48 hours after dosing.

### Statistical Analyses

Statistical analysis to determine sensor response significance was performed via two-tailed t-tests with unequal variances.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter comprising:
a single-walled carbon nanotube (SWCNT) wrapped with (1) a single-stranded DNA with 100 or fewer bases or (2) a single-stranded RNA with 100 or few bases; and
a polymer matrix comprised of 1-5% (m/m) of a cellulose material in aqueous buffer, wherein the cellulose material is methylcellulose or sulfonated methylcellulose.

2. The composition as recited in claim 1, wherein the SWCNT is present in the polymer matrix at a concentration of 0.5 mM to 500 mM.

3. The composition as recited in claim 1, wherein the SWCNT is present in the polymer matrix at a concentration of 1 mM to 100 mM.

4. The composition as recited in claim 1, wherein the SWCNT is present in the polymer matrix at a concentration of 1 mM to 10 mM.

5. The composition as recited in claim 1, wherein the cellulose material is methylcellulose.

6. The composition as recited in claim 1, wherein the cellulose material is sulfonated methylcellulose.

7. The composition as recited in claim 1, wherein the single-walled carbon nanotube (SWCNT) is wrapped with the single-stranded DNA with 50 or fewer bases.

8. The composition as recited in claim 1, wherein the single-stranded DNA is (GT)₁₅ (SEQ ID NO: 1).

9. The composition as recited in claim 1, wherein the single-walled carbon nanotube (SWCNT) is wrapped with the single-stranded RNA with 50 of fewer bases.

10. A hydrogel produced by mixing (1) a redox initiator (2) a single-walled carbon nanotube (SWCNT) that is wrapped with (1) a single-stranded DNA or (2) single-stranded RNA and (3) a polymer matrix comprised of 1-5% (m/m) of a cellulose material in aqueous buffer, wherein the cellulose material is methylcellulose or sulfonated methylcellulose.

11. The hydrogel as recited in claim 10, wherein the single-walled carbon nanotube (SWCNT) is wrapped by the single-stranded DNA with 100 or fewer bases.

12. The hydrogel as recited in claim 11, wherein the single-stranded DNA is (GT)₁₅ (SEQ ID NO: 1).

13. The hydrogel as recited in claim 10, wherein the redox initiator is ascorbic acid.

14. The hydrogel as recited in claim 10, wherein the single-walled carbon nanotube (SWCNT) is wrapped by the single-stranded RNA that has 100 or fewer bases.

15. A method comprising steps of:
injecting into an animal a two-component composition, wherein a first component comprises a redox initiator and a second component comprises a single-walled carbon nanotube (SWCNT) wrapped with (1) a single-stranded DNA or (2) a single-stranded RNA and a polymer matrix comprised of 1-5% (m/m) of a cellulose material in aqueous buffer, wherein the cellulose material is methylcellulose or sulfonated methylcellulose, the injecting occurring such that the first component mixes with the second component within the animal;
irradiating the hydrogel with electromagnetic radiation;
recording a change in intensity or a shift in wavelength of an emitted wavelength that is produced form the step of irradiating, wherein the change in intensity or the shift in wavelength is indicative of the presence of an analyte within the animal.

16. The method as recited in claim 15, wherein the single-stranded DNA has 100 or fewer bases.

17. The method as recited in claim 16, wherein the analyte is doxorubicin (DOX).

18. The method as recited in claim 16, wherein the animal is a mouse.

19. The method as recited in claim 16, wherein the animal is a human.

20. The method as recited in claim 15, wherein the single-stranded DNA is (GT)₁₅ (SEQ ID NO: 1).


SEQUENCE LISTING (1 sequence)

SEQ ID NO: 1 - DNA, 30 bp, Synthetic construct
  source 1..30: mol_type = other DNA
gtgtgtgtgtgtgtgtgtgtgtgtgtgtgt